(19) United States
(12) Patent Application Publication
(10) Pub. No.: US 2018/0297912 A1
(43) Pub. Date: Oct. 18, 2018

(54) PROCESS FOR THE OXIDATIVE COUPLING OF METHANE

(71) Applicant: SHELL OIL COMPANY, HOUSTON, TX (US)

(72) Inventors: Georgios MITKIDIS, Amsterdam (NL); Todd Paul PELTIER, Houston, TX (US); Maria SAN ROMAN MACIA, Amsterdam (NL)

(21) Appl. No.: 15/767,502

(22) PCT Filed: Oct. 14, 2016
(86) PCT No.: PCT/EP2016/074720

§ 371 Date: Apr. 11, 2018

(30) Foreign Application Priority Data
Oct. 15, 2015 (EP) 15189948.1

(51) Int. Cl. C07C 2/84; B01D 53/047; C07C 7/12; C07C 7/00; C07C 7/04

(52) U.S. Cl. CPC C07C 2/84; B01D 53/047; C07C 7/12; B01D 2256/10; C07C 7/04; B01D 2256/245; B01D 2257/7022; C07C 7/005

(57) ABSTRACT

A process for the oxidative coupling of methane to one or more C2+ hydrocarbons which includes subjecting a gas stream comprising methane, ethylene, optionally ethane and optionally nitrogen, resulting from subjecting methane and oxygen or air to methane oxidative coupling conditions, to two consecutive sorption steps which comprise contacting said gas stream comprising methane, ethylene, optionally ethane and optionally nitrogen with a selective sorption agent, resulting in sorption of part of said gas stream, comprising ethylene and optionally ethane, by the sorption agent and in a gas stream comprising the remainder of said gas stream, comprising methane, optionally ethane and optionally nitrogen, which latter gas stream is subjected to a second sorption step resulting in a further separation, finally resulting in a gas stream comprising methane, wherein each sorption step is followed by a desorption step which comprises desorbing the sorbed components originating from the gas stream in question.

### FIELD OF THE INVENTION

The present invention relates to a process for the oxidative coupling of methane.

### BACKGROUND OF THE INVENTION

Methane is a valuable resource which is used not only as a fuel, but is also used in the synthesis of chemical compounds such as higher hydrocarbons.

The conversion of methane to other chemical compounds can take place via indirect conversion wherein methane is reformed to synthesis gas (hydrogen and carbon monoxide), followed by reaction of the synthesis gas in a Fischer-Tropsch process. However, such indirect conversion is costly and consumes a lot of energy.

Consequently, it is desirable for industry to be able to convert methane directly to other chemical compounds without requiring the formation of intermediates such as synthesis gas. To this end, there has been increasing focus in recent years on the development of processes for the oxidative coupling of methane (OCM).

The oxidative coupling of methane converts methane into saturated and unsaturated, non-aromatic hydrocarbons having 2 or more carbon atoms, including ethylene. In this process, a gas stream comprising methane is contacted with an OCM catalyst and with an oxidant, such as oxygen or air. In such a process, the oxygen is adsorbed on the catalyst's surface. Methane molecules are then converted into methyl radicals. Two methyl radicals are first coupled into one ethane molecule, which is then dehydrogenated into ethylene via an ethyl radical intermediate. Said ethane and ethylene may further react into saturated and unsaturated C3+ hydrocarbons, including propane, propylene, butane, butene, etc. Usually, the gas stream leaving an OCM process contains a mixture of water, optionally hydrogen, carbon monoxide, carbon dioxide, methane, ethane, ethylene, propane, propylene, butane, butene and saturated and unsaturated hydrocarbons having 5 or more carbon atoms.

In general, the conversion that can be achieved in an OCM process is relatively low. Besides, at a higher conversion, the selectivity decreases so that it is generally desired to keep the conversion low. As a result, a relatively large amount of unconverted methane leaves the OCM reactor. The proportion of unconverted methane in the OCM product gas stream may be as high as 60 to 80 mole % based on the total molar amount of the gas stream. This unconverted methane has to be recovered from the desired products, such as ethylene and other saturated and unsaturated hydrocarbons having 2 or more carbon atoms, which are also present in such gas streams.

It is known to separate the gas stream leaving an OCM process in the following way. Acid gas (mainly CO₂) is removed in two stages, the first stage is an aqueous amine absorption system, using for example monoethanolamine (MEA), and the second stage removes final traces of CO₂ by scrubbing against aqueous NaOH. The CO₂-free gas is dried in a dessicant bed and processed in a separation train similar to that used in conventional ethylene plants. The separation sequence comprises a front end demethanizer, deethanizer, C2 splitter, depropanizer, C3 splitter, and a debutanizer. The cryogenic needs for separation are met by a propylene-ethylene cascade refrigeration system that requires ethylene refrigerant only for the demethanization stage.

Thus, it is known to separate methane from saturated and unsaturated hydrocarbons having 2 or more carbon atoms, such as ethylene, by means of cryogenic distillation in so-called “demethanizer” columns. In cryogenic distillation, a relatively high pressure and a relatively low (cryogenic) temperature are applied to effect the separation of methane. The use of cryogenic distillation following an OCM process is for example disclosed in U.S. Pat. No. 5,113,032 and U.S. Pat. No. 5,025,108.

An object of the invention is to provide a technically advantageous, efficient and affordable process for the oxidative coupling of methane, including a step wherein a product gas stream comprising (unconverted) methane and ethylene (product) is separated into a gas stream comprising the methane and another gas stream comprising the ethylene, more especially in a case where such gas stream to be separated comprises a relatively high proportion of unconverted methane. Such technically advantageous process would preferably result in a lower energy demand and/or lower capital expenditure.

### SUMMARY OF THE INVENTION

Surprisingly it was found that such technically advantageous process, resulting in a lower energy demand and/or lower capital expenditure, may be provided by subjecting a gas stream comprising methane, ethylene, optionally ethane and optionally nitrogen, resulting from subjecting methane and oxygen or air to methane oxidative coupling conditions, to two consecutive sorption steps which comprise contacting said gas stream comprising methane, ethylene, optionally ethane and optionally nitrogen with a selective sorption agent, resulting in sorption of part of said gas stream, comprising ethylene and optionally ethane, by the sorption agent and in a gas stream comprising the remainder of said gas stream, comprising methane, optionally ethane and optionally nitrogen, which latter gas stream is subjected to a second sorption step resulting in a further separation, finally resulting in a gas stream comprising methane which is optionally recycled to the methane oxidative coupling (reaction) step, wherein each sorption step is followed by a desorption step which comprises desorbing the sorbed components originating from the gas stream in question.

Accordingly, a first embodiment of the present invention relates to a process for the oxidative coupling of methane to one or more C2+ hydrocarbons, comprising

a reaction step which comprises subjecting a gas stream comprising methane and oxygen to methane oxidative coupling conditions resulting in a gas stream comprising methane, ethane and ethylene;

a first sorption step which comprises contacting the gas stream comprising methane, ethane and ethylene resulting from the reaction step with a sorption agent which has an affinity for methane which is lower than that for ethane which in turn is lower than that for ethylene, resulting in sorption of ethane and ethylene by the sorption agent and in a gas stream comprising methane and ethane;

a first desorption step which comprises desorbing ethane and ethylene sorbed in the first sorption step resulting in a gas stream comprising ethane and ethylene;

a second sorption step which comprises contacting the gas stream comprising methane and ethane resulting from the first sorption step with a sorption agent which has an affinity for methane which is lower than that for ethane, resulting in sorption of ethane by the sorption agent and in a gas stream comprising methane;

a second desorption step which comprises desorbing ethane sorbed in the second sorption step resulting in a gas stream comprising ethane; and

optionally a recycle step which comprises recycling the gas stream comprising methane resulting from the second sorption step to the reaction step.

The above-described embodiment is herein referred to as the “first embodiment” of the present invention.

Further, accordingly, a second embodiment of the present invention relates to a process for the oxidative coupling of methane to one or more C2+ hydrocarbons, comprising

a reaction step which comprises subjecting a gas stream comprising methane and air to methane oxidative coupling conditions resulting in a gas stream comprising nitrogen, methane and ethylene;

a first sorption step which comprises contacting the gas stream comprising nitrogen, methane and ethylene resulting from the reaction step with a sorption agent which has an affinity for nitrogen and methane which is lower than that for ethylene, resulting in sorption of ethylene by the sorption agent and in a gas stream comprising nitrogen and methane;

a first desorption step which comprises desorbing ethylene sorbed in the first sorption step resulting in a gas stream comprising ethylene;

a second sorption step which comprises contacting the gas stream comprising nitrogen and methane resulting from the first desorption step with a sorption agent which has an affinity for nitrogen which is lower than that for methane, resulting in sorption of methane by the sorption agent and in a gas stream comprising nitrogen;

a second desorption step which comprises desorbing methane sorbed in the second sorption step resulting in a gas stream comprising methane; and

optionally a recycle step which comprises recycling the gas stream comprising methane resulting from the second desorption step to the reaction step.

The above-described embodiment is herein referred to as the “second embodiment” of the present invention.

### BRIEF DESCRIPTION OF THE DRAWINGS

FIG. 1 shows the first embodiment of the present invention, wherein air is used as the oxidant in the reaction step and wherein the gas stream that is subjected to the first sorption step additionally comprises components other than nitrogen, methane, ethane and ethylene, namely optionally hydrogen, carbon monoxide, carbon dioxide and C3+ hydrocarbons.

### DETAILED DESCRIPTION OF THE INVENTION

Within the present specification, by “methane oxidative coupling catalyst” reference is made to a catalyst for the oxidative coupling of methane. Both terms may be used interchangeably. Analogously, by “methane oxidative coupling conditions” reference is made to conditions for the oxidative coupling of methane, which terms may also be used interchangeably.

Within the present specification, “C2+ hydrocarbons” are hydrocarbons having 2 or more carbon atoms. Likewise, within the present specification, “C3+ hydrocarbons” are hydrocarbons having 3 or more carbon atoms.

Within the present specification, by “substantially no” in relation to the amount of a specific component in a gas stream, it is meant an amount which is at most 1,000, preferably at most 500, preferably at most 100, preferably at most 50, more preferably at most 30, more preferably at most 20, and most preferably at most 10 ppmw of the component in question, based on the amount (i.e. weight) of said gas stream.

Within the present specification, where reference is made to relative (e.g. molar) amounts of components in a gas stream, such relative amounts are to be selected such that the total amount of said gas stream does not exceed 100%.

While the process of the present invention and the streams used in said process are described in terms of “comprising”, “containing” or “including” one or more various described steps and components, respectively, they can also “consist essentially of” or “consist of” said one or more various described steps and components, respectively.”.

In the reaction step of the process of the present invention, a gas stream comprising methane and an oxidant, being either oxygen (O₂) or air (which air comprises oxygen (O₂) and nitrogen (N₂)), is subjected to methane oxidative coupling conditions resulting in a gas stream comprising methane, ethane and ethylene in the first embodiment, or in a gas stream comprising nitrogen, methane and ethylene in the second embodiment.

In the above-mentioned reaction step, one gas stream comprising methane and oxygen or air may be fed to a reactor. Alternatively, two or more gas streams may be fed to the reactor, which gas streams form a combined gas stream comprising methane and oxygen or air inside the reactor. For example, one gas stream comprising oxygen or air and another gas stream comprising methane may be fed to the reactor separately.

The reactor may be any reactor suitable for the oxidative coupling of methane, such as a fixed bed reactor with axial or radial flow and with inter-stage cooling or a fluidized bed reactor equipped with internal and external heat exchangers.

Preferably, subjecting the gas stream comprising methane and oxygen or air to methane oxidative coupling conditions comprises contacting said gas stream with a methane oxidative coupling catalyst, as further described below.

In one embodiment of the present invention, a catalyst composition comprising a methane oxidative coupling catalyst may be packed along with an inert packing material, such as quartz, into a fixed bed reactor having an appropriate inner diameter and length.

Optionally, such catalyst composition may be pretreated at high temperature to remove moisture and impurities therefrom. Said pretreatment may take place, for example, at a temperature in the range of from 100-300° C. for about one hour in the presence of an inert gas such as helium.

Various processes and reactor set-ups are described in the OCM field and the process of the present invention is not limited in that regard. The person skilled in the art may conveniently employ any of such processes in the reaction step of the process of the present invention.

Suitable processes include those described in EP0206042A1, U.S. Pat. No. 4,443,649, CA2016675, U.S. Pat. No. 6,596,912, US20130023709, WO2008134484 and WO2013106771.

As used herein, the term “reactor feed” is understood to refer to the totality of the gas stream(s) at the inlet(s) of the reactor. Thus, as will be appreciated by one skilled in the art, the reactor feed is often comprised of a combination of one or more gas stream(s), such as a methane stream, an oxygen stream, an air stream, a recycle gas stream, etc.

During the oxidative coupling of methane, a reactor feed comprising methane and oxygen or air is introduced into the reactor, so that a gas stream comprising methane and oxygen or air is contacted with a methane oxidative coupling catalyst inside that reactor. Optionally, the reactor feed may further comprise minor components of the methane feed (ethane, propane etc.) or the methane recycle stream (e.g. ethane, ethylene, propane, propylene, CO, CO₂, N₂, H₂ and H₂O).

In a case wherein in the present invention oxygen (not air) is used as the oxidant in the reaction step, the gas stream comprising oxygen (to be combined with the methane in the reaction step), may be a high purity oxygen stream. Such high-purity oxygen may have a purity greater than 90%, preferably greater than 95%, more preferably greater than 99%, and most preferably greater than 99.4%.

In the reaction step of the process of the present invention, methane and oxygen or air may be added to the reactor as mixed feed, optionally comprising further components therein, at the same reactor inlet. Alternatively, the methane and oxygen or air may be added in separate feeds, optionally comprising further components therein, to the reactor at the same reactor inlet or at separate reactor inlets.

In the reaction step of the process of the present invention, the methane:oxygen molar ratio (wherein the oxygen may or may not originate from air) in the reactor feed may be in the range of from 2:1 to 10:1, more preferably 3:1 to 6:1. In a case wherein in the present invention air is used as the oxidant in the reaction step, such methane:oxygen molar ratios correspond to methane:air molar ratios of 2:4.8 to 10:4.8 and 3:4.8 to 6:4.8, respectively.

In a case wherein in the present invention air is used as the oxidant in the reaction step, methane may be present in the reactor feed in a concentration of at least 35 mole %, more preferably at least 40 mole %, relative to the reactor feed. Further, methane may be present in the reactor feed in a concentration of at most 90 mole %, more preferably at most 85 mole %, most preferably at most 80 mole %, relative to the reactor feed. Thus, in the present invention, methane may for example be present in the reactor feed in a concentration in the range of from 35 to 90 mole %, more preferably 40 to 85 mole %, most preferably 40 to 80 mole %, relative to the reactor feed.

In general, the oxygen concentration (wherein the oxygen may or may not originate from air) in the reactor feed should be less than the concentration of oxygen that would form a flammable mixture at either the reactor inlet or the reactor outlet at the prevailing operating conditions.

In a case wherein in the present invention air is used as the oxidant in the reaction step, air may be present in the reactor feed in a concentration of at least 10 mole %, more preferably at least 15 mole %, most preferably at least 20 mole %, relative to the reactor feed. Further, air may be present in the reactor feed in a concentration of at most 65 mole %, more preferably at most 60 mole %, relative to the reactor feed. Thus, in said embodiment, air may for example be present in the reactor feed in a concentration in the range of from 10 to 65 mole %, more preferably 15 to 60 mole %, most preferably 20 to 60 mole %, relative to the reactor feed.

In a case wherein in the present invention oxygen (not air) is used as the oxidant in the reaction step, suitable ethane and oxygen concentrations in the reactor feed can be calculated from the above-mentioned ethane and air concentrations in the reactor feed, taking into account that air comprises 21 mole % of oxygen. Thus, oxygen may be present in the reactor feed in a concentration of at least 2.1 mole %, more preferably at least 3.2 mole %, most preferably at least 4.2 mole %, relative to the reactor feed. Further, oxygen may be present in the reactor feed in a concentration of at most 13.7 mole %, more preferably at most 12.6 mole %, relative to the reactor feed. Thus, in said embodiment, oxygen may for example be present in the reactor feed in a concentration in the range of from 2.1 to 13.7 mole %, more preferably 3.2 to 12.6 mole %, most preferably 4.2 to 12.6 mole %, relative to the reactor feed.

In the above-mentioned reaction step, a reactor feed comprising methane and oxygen or air is subjected to methane oxidative coupling conditions, which as discussed above, may comprise contacting said gas stream with a methane oxidative coupling catalyst so that methane is converted to one or more C2+ hydrocarbons. Suitably, the reactor temperature in said reaction step is in the range of from 500 to 1000° C. Preferably, said conversion is effected at a reactor temperature in the range of from 700 to 1000° C., more preferably in the range of from 750 to 900° C.

In a preferred embodiment, said conversion of methane to one or more C2+ hydrocarbons is effected at a reactor pressure in the range of from 2 to 20 bar, more preferably 5 to 15 bar.

According to the present invention, the above-mentioned methane oxidative coupling catalyst may be any methane oxidative coupling catalyst. Generally, the catalyst may contain one or more of manganese, one or more alkali metals (e.g. sodium) and tungsten. Preferably, the catalyst contains manganese, one or more alkali metals (e.g. sodium) and tungsten. Said carrier may be unsupported or supported. In particular, the catalyst may be a mixed metal oxide catalyst containing manganese, one or more alkali metals (e.g. sodium) and tungsten. Further, the catalyst may be a supported catalyst, such as a catalyst comprising manganese, one or more alkali metals (e.g. sodium) and tungsten on a carrier. The carrier may be any carrier, such as silica or a metal-containing carrier. A particular suitable catalyst comprises manganese, tungsten and sodium on a silica carrier (Mn—Na₂WO₄/SiO₂).

Suitable methane oxidative coupling catalysts are described in the following publications.

Chua et al. studied the oxidative coupling of methane for the production of ethylene over sodium-tungsten-manganese-supported silica catalyst (Na—W—Mn/SiO₂) in Applied Catalysis A: General 343 (2008) 142-148.

The performance of Mn—Na₂WO₄/SiO₂ catalyst was further reviewed by Arndt et al. in Applied Catalysis A: General 425-426 (2012) 53-61 and Lee et al. in Fuel 106 (2013) 851-857.

US20130023709 describes the high throughput screening of catalyst libraries for the oxidative coupling of methane and tests various catalysts including catalysts comprising sodium, manganese and tungsten on silica and zirconia carriers.

US20140080699 describes a specific method for the preparation of catalysts such as Mn—Na₂WO₄/SiO₂ catalyst which is said to provide an improved catalyst material.

Various manganese and titanium-containing catalysts for the oxidative coupling of methane are researched in the literature and are disclosed in various patent publications including Gong et al. Catalysis Today 24 (1995), 259-261, Gong et al. Catalysis Today 24 (1995), 263-264, Jeon et al. Applied Catalysis A: General 464-465 (2013) 68-77, U.S. Pat. No. 4,769,508 and US20130178680.

In general, the gas stream comprising methane, ethylene, optionally ethane and optionally nitrogen resulting from the above-described reaction step also comprises water. Water may easily be removed from said gas stream, for example by cooling down the gas stream from the reaction temperature to a lower temperature, for example room temperature, so that the water condenses and can then be removed from the gas stream. Therefore, preferably, in an embodiment wherein the gas stream resulting from the above-described reaction step comprises methane, ethylene, optionally ethane, optionally nitrogen and water, water is removed from such gas stream in the above-mentioned way, preferably before the below-described first sorption step is carried out.

Such condensing step, as described above, may be followed by a drying step in order to remove substantially all water, also preferably before the below-described first sorption step is carried out. For example, such drying may be carried out by contacting the gas stream with an absorption agent which has a high affinity for water, such as for example triethylene glycol (TEG), for example at a temperature in the range of from 30 to 50° C., suitably about 40° C. Alternatively, such drying may be carried out by contacting the gas stream with molecular sieves (or “mol sieves”), suitably at a relatively low temperature in the range of from 10 to 25° C. Using molecular sieves is preferred in a case where the remaining water content should be as low as possible.

The removal of water before the below-described first sorption step, as described above, is preferred because then advantageously less sorption agent may be used in the latter step since there is less or substantially no water to be sorbed by the sorption agent. Further, by removing water at this stage, advantageously less or substantially no water will interfere with downstream purification of gas streams coming from the below-described sorption steps and/or desorption steps.

In the first embodiment of the present invention, the gas stream comprising methane, ethane and ethylene resulting from the above-described reaction step is subjected to a first sorption step. Suitably, the gas stream that is subjected to that first sorption step comprises 20 to 70 mole % of methane, more suitably 25 to 65 mole % of methane; 0.02 to 6 mole % of ethane, more suitably 0.1 to 6 mole % of ethane; 0.06 to 12 mole % of ethylene, more suitably 0.3 to 12 mole % of ethylene; and 0 to 10 mole % of oxygen, more suitably 0 to 5 mole % of oxygen. Said relative amounts are based on the total amount of the gas stream.

In the second embodiment of the present invention, the gas stream comprising nitrogen, methane and ethylene resulting from the above-described reaction step is subjected to a first sorption step. Suitably, the gas stream that is subjected to that first sorption step comprises 25 to 56 mole % of nitrogen, more suitably 30 to 50 mole % of nitrogen; 20 to 70 mole % of methane, more suitably 25 to 65 mole % of methane; 0.1 to 15 mole % of ethylene, more suitably 0.5 to 15 mole % of ethylene; and 0 to 10 mole % of oxygen, more suitably 0 to 5 mole % of oxygen. Said relative amounts are based on the total amount of the gas stream.

In the first embodiment of the present invention, in the first sorption step, the gas stream comprising methane, ethane and ethylene resulting from the reaction step is contacted with a sorption agent which has an affinity for methane which is lower than that for ethane which in turn is lower than that for ethylene, resulting in sorption of ethane and ethylene by the sorption agent and in a gas stream comprising methane and ethane. That is to say, the gas stream resulting from the first sorption step comprises methane and ethane that are not sorbed by the sorption agent. In particular, the amount of ethane in the gas stream resulting from the first sorption step is greater than 0% to smaller than 100%, based on the amount of ethane in the gas stream that is subjected to the first sorption step. The latter percentage may also be referred to as “ethane rejection” (ethane not being sorbed, but “rejected”). Such “ethane rejection” may be varied by varying the pressure, temperature, nature of the sorption agent and/or configuration of the sorption-desorption system.

In the first desorption step of the first embodiment of the present invention, ethane and ethylene that are sorbed by the sorption agent are desorbed, resulting in a gas stream comprising ethane and ethylene. That is to say, the latter gas stream resulting from the first desorption step comprises ethane and ethylene that are desorbed from the sorption agent.

Further, in a second sorption step of the first embodiment of the present invention, the gas stream comprising methane and ethane resulting from the first sorption step is contacted with a sorption agent which has an affinity for methane which is lower than that for ethane, resulting in sorption of ethane by the sorption agent and in a gas stream comprising methane. In the second desorption step of the first embodiment, ethane that is sorbed by the sorption agent is desorbed, resulting in a gas stream comprising ethane. Advantageously, ethane from said gas stream comprising ethane may be converted into ethylene, for example by steam cracking, thereby increasing the total yield of ethylene. Optionally, the first embodiment comprises a recycle step which comprises recycling the gas stream comprising methane resulting from the second sorption step to the reaction step.

In the first embodiment of the present invention, oxygen may be fed to the reaction step in the form of air, whereas in the second embodiment of the present invention, oxygen should be fed to the reaction step in the form of air. Said second embodiment will be further described hereinbelow. Therefore, in the first embodiment, the oxidant in the reaction step may be air or an oxidant other than air, such as for example the above-described high-purity oxygen.

Thus, as described above, in the first embodiment of the present invention, the oxidant in the reaction step may be air. In that case, said first embodiment comprises

a reaction step which comprises subjecting a gas stream comprising methane and air to methane oxidative coupling conditions resulting in a gas stream comprising nitrogen, methane, ethane and ethylene;

a first sorption step which comprises contacting the gas stream comprising nitrogen, methane, ethane and ethylene resulting from the reaction step with a sorption agent which has an affinity for nitrogen which is lower than that for methane which in turn is lower than that for ethane which in turn is lower than that for ethylene, resulting in sorption of ethane and ethylene by the sorption agent and in a gas stream comprising nitrogen, methane and ethane;

a first desorption step which comprises desorbing ethane and ethylene sorbed in the first sorption step resulting in a gas stream comprising ethane and ethylene;

a second sorption step which comprises contacting the gas stream comprising nitrogen, methane and ethane resulting from the first sorption step with a sorption agent which has an affinity for nitrogen which is lower than that for methane which in turn is lower than that for ethane, resulting in sorption of ethane by the sorption agent and in a gas stream comprising nitrogen and methane;

a second desorption step which comprises desorbing ethane sorbed in the second sorption step resulting in a gas stream comprising ethane.

In a case, wherein in the first embodiment of the present invention, the oxidant in the reaction step is air, preferably, said first embodiment additionally comprises

a third sorption step which comprises contacting the gas stream comprising nitrogen and methane resulting from the second sorption step with a sorption agent which has an affinity for nitrogen which is lower than that for methane, resulting in sorption of methane by the sorption agent and in a gas stream comprising nitrogen;

a third desorption step which comprises desorbing methane sorbed in the third sorption step resulting in a gas stream comprising methane; and

optionally a recycle step which comprises recycling the gas stream comprising methane resulting from the third desorption step to the reaction step.

In the second embodiment of the present invention, in the first sorption step, the gas stream comprising nitrogen, methane and ethylene resulting from the reaction step is contacted with a sorption agent which has an affinity for nitrogen and methane which is lower than that for ethylene, resulting in sorption of ethylene by the sorption agent and in a gas stream comprising nitrogen and methane. That is to say, the gas stream resulting from the first sorption step comprises nitrogen and methane that are not sorbed by the sorption agent. In particular, the amount of methane in the gas stream resulting from the first sorption step is greater than 0% to 100%, based on the amount of methane in the gas stream that is subjected to the first sorption step. The latter percentage may also be referred to as “methane rejection” (methane not being sorbed, but “rejected”). Such “methane rejection” may be varied by varying the pressure, temperature, nature of the sorption agent and/or configuration of the sorption-desorption system.

In the first desorption step of the second embodiment of the present invention, ethylene that is sorbed by the sorption agent is desorbed, resulting in a gas stream comprising ethylene. That is to say, the latter gas stream resulting from the desorption step comprises ethylene that is desorbed from the sorption agent.

Further, in a second sorption step of the second embodiment of the present invention, the gas stream comprising nitrogen and methane resulting from the first sorption step is contacted with a sorption agent which has an affinity for nitrogen which is lower than that for methane, resulting in sorption of methane by the sorption agent and in a gas stream comprising nitrogen. In the second desorption step of the second embodiment, methane that is sorbed by the sorption agent is desorbed, resulting in a gas stream comprising methane. Optionally, the second embodiment comprises a recycle step which comprises recycling the gas stream comprising methane resulting from the second desorption step to the reaction step.

In the first and second sorption steps of the process of the present invention, sorption agents are used. In the present specification, “sorption” means a process in which one substance (the sorption agent) takes up or holds or retains another substance by absorption, adsorption or a combination of both.

Further, a sorption agent used in a sorption step of the first or second embodiment of the present invention should have a certain affinity (selectivity). This will be demonstrated hereinbelow with reference to the sorption agent to be used in the first sorption step of the first embodiment of the present invention, but similar principles apply to the sorption agents used in the second sorption step of the first embodiment of the present invention and in the sorption steps of the second embodiment of the present invention. In said first sorption step of the first embodiment, the sorption agent has an affinity for methane which is lower than that for ethane which in turn is lower than that for ethylene. This means that under the conditions applied in said sorption step, including pressure and temperature which are further defined hereinbelow, said sorption agent has an affinity for methane which is lower than that for ethane which in turn is lower than that for ethylene. This implies that such sorption agent should be used, that the molar ratio of sorbed ethylene to sorbed methane is greater than 1:1, assuming equal partial pressures for ethylene, ethane and methane. Preferably, said ratio is equal to or higher than 1.1:1, more preferably equal to or higher than 5:1. Said ratio may be up to 50:1, or may be up to 40:1, or may be up to 30:1, or may be up to 20:1. For example, said ratio is in the range of from 1.1:1 to 50:1 or from 5:1 to 20:1. Sorption agents suitable to be used in the first sorption step of the first embodiment may be selected by comparing the extent of sorption of methane, the extent of sorption of ethane and the extent of sorption of ethylene, under any given temperature and pressure conditions for a variety of known sorption agents, assuming equal partial pressures for ethylene and methane. Therefore, a wide range of sorption agents may be used since the only criterion for the sorption agent to be used in the first sorption step of the first embodiment is that it should have an affinity for methane which is lower than that for ethane which in turn is lower than that for ethylene.

Without any limitation, examples of suitable sorption agents are activated carbons; molecular sieves and zeolites (e.g. zeolite 13X, zeolite 5A, ZSM-5, SAPO-34); mesoporous silicas (e.g. SBA-2, SBA-15); porous silicas (e.g. CMK-3, silicate-1); clay heterostructures; Engelhard Titanosilicates (ETS; e.g. ETS-4, ETS-10); porous coordination polymers (PCPs); cation impregnated porous adsorbents and zeolites (e.g. AgA); metal-organic frameworks (MOFs); Zeolitic Imidazolate Framework (ZIFs); and Carbon Organic Frameworks (COFs). Suitable sorption agents are for example disclosed in US20150065767 and US20140249339.

In the sorption steps of the first and second embodiments of the present invention (first, second and optional third sorption steps), the same or a different sorption agent may be used. A different sorption agent (having a different selectivity) may be used in a case where the pressure in these sorption steps is the same, for example in a case where the gas stream resulting from the first sorption step is sent to the second sorption step without any further compression, as described below. Alternatively or additionally, in such case, the temperature and/or configuration of the sorption-desorption system may be varied. In a case where the same sorption agent is used, the pressure, temperature and/or configuration of the sorption-desorption system may be varied in order to achieve the different selective sorption.

The pressure in the sorption steps of the first and second embodiments of the present invention (first, second and optional third sorption steps) may vary within wide ranges. Preferably, said pressure is equal to or higher than atmospheric pressure and at most 30 bar, more preferably at most 15 bar. More preferably, said pressure is of from 5 to 30 bar, more preferably 5 to 15 bar, most preferably 7 to 13 bar. In the reaction step of the process of the present invention, oxygen or air may be fed at a pressure in the range of from 5 to 30 bar, preferably 5 to 15 bar, more preferably 7 to 13 bar. This implies advantageously that the gas stream resulting from the reaction step generally need not be compressed before subjecting it to the first sorption step. Thus, preferably, the pressure at which oxygen or air is fed in the reaction step is the same as the pressure in the first sorption step.

The temperature in the sorption steps of the first and second embodiments of the present invention (first, second and optional third sorption steps) may also vary within wide ranges. Preferably, said temperature is in the range of from 0 to 100° C., more preferably 10 to 90° C., most preferably 25 to 80° C. Advantageously, in the present invention, said sorption steps may be carried out at a non-cryogenic temperature (e.g. of from 0 to 100° C. as mentioned above).

Preferably, in the desorption steps of the first and second embodiments of the present invention (first, second and optional third desorption steps), desorption is effected by reducing the pressure. That is to say, the pressure in a certain desorption step (for example first desorption step) is lower than the pressure in the sorption step that directly precedes said desorption step (for example first sorption step). This is usually referred to as “Pressure Swing Adsorption” (PSA). In the embodiments wherein desorption in said desorption steps is effected by reducing the pressure, the pressure in the sorption steps (first, second and optional third sorption steps) is preferably in the range of from 5 to 30 bar, more preferably 5 to 15 bar, more preferably 7 to 13 bar.

Advantageously, in the sorption steps of the first and second embodiments of the present invention (first, second and optional third sorption steps), a relatively low pressure may be applied (e.g. of from 5 to 15 bar as mentioned above). Such low pressure advantageously results in that relatively less compression of the gas stream may be needed. It is especially advantageous that the pressure that may be needed in said sorption steps may be the same as the pressure in the (OCM) reaction step. In the latter case, there would be no need at all for any compression of said gas stream in order to carry out the sorption steps.

A further advantage of the present invention is that at least two sorption steps are carried out. In the present invention, this implies advantageously that the gas stream resulting from the first sorption step may be sent to the second sorption step without any further compression. Optionally and where needed, as described above, the gas stream resulting from the second sorption step may be sent to a third sorption step also without any further compression.

Further, in the desorption steps of the first and second embodiments of the present invention (first, second and optional third desorption steps), wherein desorption in the desorption steps is effected by reducing the pressure, the pressure in the desorption steps is preferably in the range of from 0.1 to 3 bar, more preferably 0.5 to 2 bar.

The temperature in the desorption steps of the first and second embodiments of the present invention (first, second and optional third desorption steps) may also vary within wide ranges. Preferably, said temperature is in the range of from 0 to 100° C., more preferably 10 to 90° C., most preferably 25 to 80° C. Advantageously, in the present invention, said desorption steps may be carried out at a non-cryogenic temperature (e.g. of from 0 to 100° C. as mentioned above).

Advantageously, the sorption and desorption steps of the process of the present invention make it possible to first efficiently separate methane, optionally ethane and optionally nitrogen from a gas stream comprising methane, ethylene, optionally ethane and optionally nitrogen resulting from the (OCM) reaction step, and then efficiently separate methane from a gas stream comprising methane and ethane resulting from the first sorption step (first embodiment of the present invention) or from a gas stream comprising nitrogen and methane resulting from the first sorption step (second embodiment of the present invention), at a relatively low pressure (e.g. at most 15 bar as mentioned above) and at a non-cryogenic temperature (e.g. of from 0 to 100° C. as mentioned above).

Preferably, the gas stream comprising methane, ethylene, optionally ethane and optionally nitrogen that is subjected to the first sorption step of the process of the present invention comprises substantially no water. As described above, preferably, any water is removed from said gas stream before the first sorption step is carried out. It is also preferred that said gas stream comprising methane, ethylene, optionally ethane and optionally nitrogen comprises substantially no hydrogen sulfide.

In the first embodiment of the present invention, wherein the first sorption step results in sorption of ethane and ethylene by the sorption agent, and the desorption step comprises desorbing sorbed ethane and ethylene resulting in a gas stream comprising ethane and ethylene, preferably, the first embodiment additionally comprises a distillation step which comprises distilling the gas stream comprising ethane and ethylene resulting from the first desorption step, resulting in a top stream comprising ethylene and a bottom stream comprising ethane.

As is demonstrated in the present Examples, it has surprisingly appeared that advantageously the energy demand, especially the demand for compression and refrigeration energy, is significantly lower as compared to a process wherein a sorption and desorption method, comprising multiple sorption and desorption steps, is not applied after the OCM reaction step. Thus, the present process is a process that enables the oxidative coupling of methane and subsequent separation of the product stream comprising methane, ethylene, optionally ethane and optionally nitrogen, to recover unconverted methane and ethylene, in a way that is technically feasible, efficient and affordable since the energy demand is surprisingly lower as compared to the comparative process.

Further, in the first and second embodiments of the present invention, the gas stream comprising methane, ethylene, optionally ethane and optionally nitrogen that is subjected to the first sorption step of the process of the present invention additionally comprises components other than said methane, ethylene, ethane and nitrogen, such as carbon monoxide, optionally hydrogen, carbon dioxide and C3+ hydrocarbons. Therefore, in the present invention, the reaction step may result in a gas stream comprising methane, ethylene, optionally hydrogen, optionally nitrogen, carbon monoxide, carbon dioxide, ethane and C3+ hydrocarbons.

Suitably, said C3+ hydrocarbons comprise saturated and unsaturated C3+ hydrocarbons, including propane, propylene, butane and butene, and optionally saturated and unsaturated hydrocarbons having 5 or more carbon atoms.

In a case wherein in the first embodiment of the present invention, the reaction step results in a gas stream comprising methane, ethylene, optionally hydrogen, optionally nitrogen, carbon monoxide, carbon dioxide, ethane and C3+ hydrocarbons,

the first sorption step comprises contacting the gas stream comprising methane, ethylene, optionally hydrogen, optionally nitrogen, carbon monoxide, carbon dioxide, ethane and C3+ hydrocarbons resulting from the reaction step with a sorption agent which has an affinity for hydrogen, nitrogen, carbon monoxide and methane which is lower than that for ethane which in turn is lower than that for carbon dioxide, ethylene and C3+ hydrocarbons, resulting in sorption of carbon dioxide, ethane, ethylene and C3+ hydrocarbons by the sorption agent and in a gas stream comprising optionally hydrogen, optionally nitrogen, carbon monoxide, methane and ethane;

the first desorption step comprises desorbing sorbed carbon dioxide, ethane, ethylene and C3+ hydrocarbons resulting in a gas stream comprising carbon dioxide, ethane, ethylene and C3+ hydrocarbons;

the second sorption step comprises contacting the gas stream comprising optionally hydrogen, optionally nitrogen, carbon monoxide, methane and ethane resulting from the first sorption step with a sorption agent which has an affinity for hydrogen, nitrogen, carbon monoxide and methane which is lower than that for ethane, resulting in sorption of ethane by the sorption agent and in a gas stream comprising optionally hydrogen, optionally nitrogen, carbon monoxide and methane; and

the second desorption step comprises desorbing ethane sorbed in the second sorption step resulting in a gas stream comprising ethane.

In a case, wherein in the first embodiment of the present invention, the gas stream resulting from the second sorption step comprises optionally hydrogen, optionally nitrogen, carbon monoxide and methane, preferably, said first embodiment additionally comprises

a third sorption step which comprises contacting the gas stream comprising optionally hydrogen, optionally nitrogen, carbon monoxide and methane resulting from the second sorption step with a sorption agent which has an affinity for hydrogen, nitrogen and carbon monoxide which is lower than that for methane, resulting in sorption of methane by the sorption agent and in a gas stream comprising optionally hydrogen, optionally nitrogen and carbon monoxide;

a third desorption step which comprises desorbing methane sorbed in the third sorption step resulting in a gas stream comprising methane; and

optionally a recycle step which comprises recycling the gas stream comprising methane resulting from the third desorption step to the reaction step.

In a case wherein in the second embodiment of the present invention, the reaction step results in a gas stream comprising methane, ethylene, optionally hydrogen, nitrogen, carbon monoxide, carbon dioxide, ethane and C3+ hydrocarbons,

the first sorption step comprises contacting the gas stream comprising methane, ethylene, optionally hydrogen, nitrogen, carbon monoxide, carbon dioxide, ethane and C3+ hydrocarbons resulting from the reaction step with a sorption agent which has an affinity for hydrogen, nitrogen, carbon monoxide and methane which is lower than that for carbon dioxide, ethane, ethylene and C3+ hydrocarbons, resulting in sorption of carbon dioxide, ethane, ethylene and C3+ hydrocarbons by the sorption agent and in a gas stream comprising hydrogen, nitrogen, carbon monoxide and methane;

the first desorption step comprises desorbing carbon dioxide, ethane, ethylene and C3+ hydrocarbons sorbed in the first sorption step resulting in a gas stream comprising carbon dioxide, ethane, ethylene and C3+ hydrocarbons;

the second sorption step comprises contacting the gas stream comprising hydrogen, nitrogen, carbon monoxide and methane resulting from the first desorption step with a sorption agent which has an affinity for hydrogen, nitrogen and carbon monoxide which is lower than that for methane, resulting in sorption of methane by the sorption agent and in a gas stream comprising hydrogen, nitrogen and carbon monoxide; and

the second desorption step comprises desorbing methane sorbed in the second sorption step resulting in a gas stream comprising methane.

The sorption agents, pressures, temperatures, sorption-desorption method (e.g. PSA) and configuration of the sorption-desorption system as discussed above also apply to the above-mentioned embodiments of the present invention, wherein the gas stream that is subjected to the first sorption step (gas stream resulting from the reaction step) comprises methane, ethylene, optionally hydrogen, optionally nitrogen, carbon monoxide, carbon dioxide, ethane and C3+ hydrocarbons.

Further, preferably, in the above-mentioned first and second embodiments, the process of the present invention additionally comprises a carbon dioxide removal step which comprises removing carbon dioxide from the gas stream comprising carbon dioxide, ethane, ethylene and C3+ hydrocarbons resulting from the first desorption step, resulting in a gas stream comprising ethylene, ethane and C3+ hydrocarbons. In said carbon dioxide removal step, carbon dioxide may be removed by any known method, such as treatment with an amine and then with a caustic agent, such as an aqueous monoethanolamine (MEA) absorption system and aqueous NaOH, respectively, as already mentioned above in the introduction of this specification. In a case where said carbon dioxide removal step involves the use of water, said step normally also involves the removal of that water, suitably followed by a drying step. Such drying step may be carried out in order to remove substantially all water and may be carried out in one of the ways as exemplified above in relation to the optional drying step after a condensing step.

The above-described first and second embodiments of the present invention, comprising the first and second and optional third sorption and desorption steps followed by the carbon dioxide removal step, may additionally comprise a first distillation step wherein the gas stream comprising ethylene, ethane and C3+ hydrocarbons resulting from the carbon dioxide removal step as carried out after the first desorption step is distilled. Preferably, said first distillation step comprises distilling the gas stream comprising ethylene, ethane and C3+ hydrocarbons resulting from the carbon dioxide removal step, resulting in a top stream comprising ethylene and ethane and a bottom stream comprising C3+ hydrocarbons. Further, the process of the present invention may additionally comprise a second distillation step which comprises distilling the above-mentioned top stream comprising ethylene and ethane, resulting in a top stream comprising ethylene and a bottom stream comprising ethane. Further, said first distillation step may comprise distilling the gas stream comprising ethylene, ethane and C3+ hydrocarbons resulting from the carbon dioxide removal step, resulting in a top stream comprising ethylene and a bottom stream comprising ethane and C3+ hydrocarbons. Further, the process of the present invention may additionally comprise a second distillation step which comprises distilling the above-mentioned bottom stream comprising ethane and C3+ hydrocarbons, resulting in a top stream comprising ethane and a bottom stream comprising C3+ hydrocarbons.

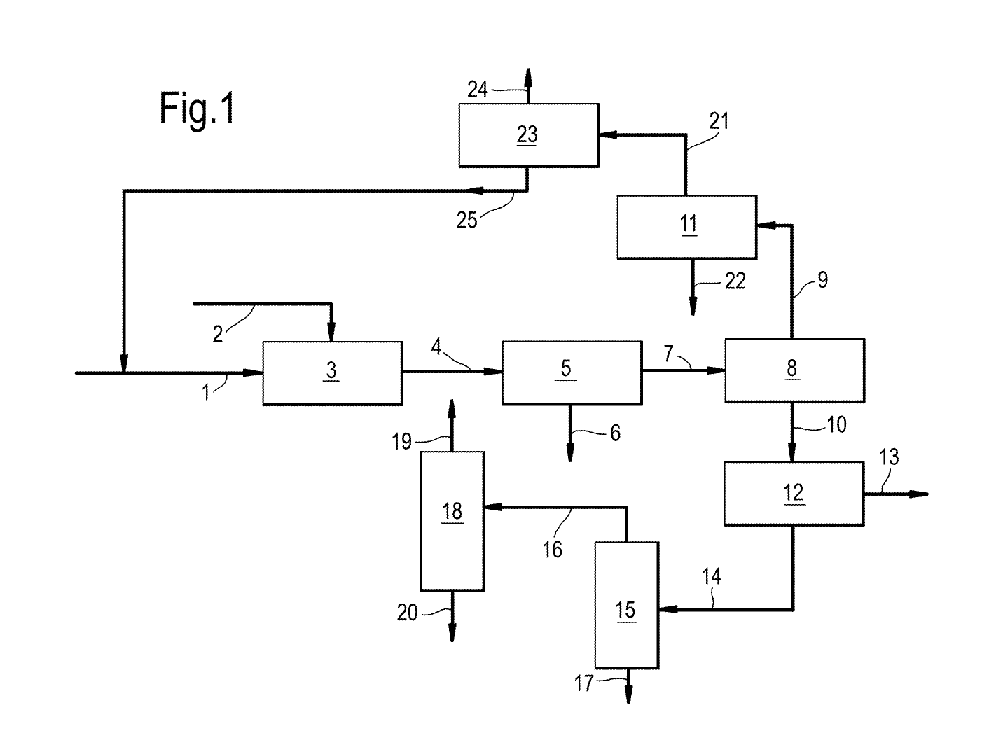

An example of the first embodiment of the present invention, wherein air is used as the oxidant in the reaction step and wherein the gas stream that is subjected to the first sorption step additionally comprises components other than nitrogen, methane, ethane and ethylene, namely optionally hydrogen, carbon monoxide, carbon dioxide and C3+ hydrocarbons, is schematically shown in FIG. 1. Hereinafter, the combination of ethylene, ethane and C3+ hydrocarbons may also be referred to as C2+ hydrocarbons.

In said FIG. 1, a gas stream 1 comprising methane and an air stream 2 are fed to a methane oxidative coupling (OCM) reactor 3 containing an OCM catalyst and operating under OCM conditions. Product stream 4 originating from OCM reactor 3 comprises water, methane, ethylene, hydrogen, nitrogen, carbon monoxide, carbon dioxide, ethane and C3+ hydrocarbons. Said stream 4 is fed to condensation vessel 5 where water is removed via stream 6. Gas stream 7 comprising methane, ethylene, hydrogen, nitrogen, carbon monoxide, carbon dioxide, ethane and C3+ hydrocarbons originating from condensation vessel 5 is fed to sorption and desorption unit 8. Optionally, before gas stream 7 is fed to sorption and desorption unit 8, it is sent to a drying unit (not shown in FIG. 1) in order to remove substantially all water.

Sorption and desorption unit 8 comprises a sorption agent which has an affinity for hydrogen, nitrogen, carbon monoxide and methane which is lower than that for ethane which in turn is lower than that for carbon dioxide, ethylene and C3+ hydrocarbons. The pressure of gas stream 7 may be of from 5 to 15 bar. Carbon dioxide, ethane, ethylene and C3+ hydrocarbons are sorbed by the sorption agent. Further, a gas stream 9 comprising nitrogen, hydrogen, carbon monoxide, methane and ethane leaves sorption and desorption unit 8, which nitrogen, hydrogen, carbon monoxide, methane and ethane are not sorbed by the sorption agent in sorption and desorption unit 8. Gas stream 9 is sent to sorption and desorption unit 11.

After some time, the feed of gas stream 7 to sorption and desorption unit 8 is stopped and the pressure in said unit is reduced. For example, the pressure in sorption and desorption unit 8 may be reduced to a pressure in the range of from 0.1 to 3 bar in a case wherein during the sorption step the pressure is in the range of from 5 to 15 bar, as exemplified above. Through such pressure reduction carbon dioxide and C2+ hydrocarbons that are sorbed by the sorption agent become desorbed. A gas stream 10 comprising carbon dioxide and C2+ hydrocarbons, that are desorbed from the sorption agent, leaves sorption and desorption unit 8 and is sent to carbon dioxide removal unit 12.

Once the desorption is completed, the feed of gas stream 7 to sorption and desorption unit 8 is resumed and the above procedure is repeated.

Sorption and desorption unit 11 comprises a sorption agent which has an affinity for hydrogen, nitrogen, carbon monoxide and methane which is lower than that for ethane. The pressure of gas stream 9 may be of from 5 to 15 bar. Ethane is sorbed by the sorption agent. Further, a gas stream 21 comprising nitrogen, hydrogen, carbon monoxide and methane leaves sorption and desorption unit 11, which nitrogen, hydrogen, carbon monoxide and methane are not sorbed by the sorption agent in sorption and desorption unit 11. Gas stream 21 is sent to sorption and desorption unit 23.

After some time, the feed of gas stream 9 to sorption and desorption unit 11 is stopped and the pressure in said unit is reduced. For example, the pressure in sorption and desorption unit 11 may be reduced to a pressure in the range of from 0.1 to 3 bar in a case wherein during the sorption step the pressure is in the range of from 5 to 15 bar, as exemplified above. Through such pressure reduction ethane that is sorbed by the sorption agent becomes desorbed. A gas stream 22 comprising ethane that is desorbed from the sorption agent, leaves sorption and desorption unit 11. Ethane from gas stream 22 may be converted into ethylene, for example by steam cracking.

Once the desorption is completed, the feed of gas stream 9 to sorption and desorption unit 11 is resumed and the above procedure is repeated.

Sorption and desorption unit 23 comprises a sorption agent which has an affinity for hydrogen, nitrogen and carbon monoxide which is lower than that for methane. The pressure of gas stream 9 may be of from 5 to 15 bar. Methane is sorbed by the sorption agent. Further, a gas stream 24 comprising nitrogen, hydrogen and carbon monoxide leaves sorption and desorption unit 23, which nitrogen, hydrogen and carbon monoxide are not sorbed by the sorption agent in sorption and desorption unit 23.

After some time, the feed of gas stream 21 to sorption and desorption unit 23 is stopped and the pressure in said unit is reduced. For example, the pressure in sorption and desorption unit 23 may be reduced to a pressure in the range of from 0.1 to 3 bar in a case wherein during the sorption step the pressure is in the range of from 5 to 15 bar, as exemplified above. Through such pressure reduction methane that is sorbed by the sorption agent becomes desorbed. A gas stream 25 comprising methane that is desorbed from the sorption agent, leaves sorption and desorption unit 23 and is recycled to OCM reactor 3.

Once the desorption is completed, the feed of gas stream 21 to sorption and desorption unit 23 is resumed and the above procedure is repeated.

In carbon dioxide removal unit 12, carbon dioxide is removed, via stream 13, from gas stream 10 comprising carbon dioxide and C2+ hydrocarbons, in a way as exemplified above, that is to say involving the use of water and the removal of that water. A gas stream 14 comprising C2+ hydrocarbons leaves carbon dioxide removal unit 12. Before gas stream 14 is sent to distillation column 15, it is sent to a drying unit (not shown in FIG. 1) in order to remove substantially all water.

In distillation column 15, gas stream 14 comprising C2+ hydrocarbons is distilled such that separation between on the one hand ethylene and ethane and on the other hand C3+ hydrocarbons is effected. That is, a top stream 16 comprising ethylene and ethane and a bottom stream 17 comprising C3+ hydrocarbons leave distillation column 15.

Top stream 16 comprising ethylene and ethane is sent to distillation column 18, wherein it is distilled such that separation between ethylene and ethane is effected. That is, a top stream 19 comprising ethylene and a bottom stream 20 comprising ethane leave distillation column 18.

If in the setup of FIG. 1, a gas stream 1 comprising methane of sufficiently high pressure (for example in the range of from 5 to 15 bar, which may be the case for a natural gas stream) is fed to OCM reactor 3, gas compressors would advantageously only be needed in line 2 (compression of air); in line 10 (compression of gas stream 10 leaving sorption and desorption unit 8, after desorption, and entering carbon dioxide removal unit 12); and in line 25 (compression of gas stream 25 leaving sorption and desorption unit 23, after desorption, and entering OCM reactor 3).

The invention is further illustrated by the following Examples.

### Examples A and C and Comparative Examples B and D

In Example A exemplifying the 1^{st} embodiment of the present invention and in Comparative Example B, a gas stream comprising methane having a temperature of 40° C. and a pressure of 10 bar (said gas stream originating from a natural gas source) is fed to a methane oxidative coupling (OCM) reactor. In addition, a gas stream comprising oxygen, and having a temperature of 40° C. and a pressure of 10 bar, is fed to the OCM reactor.

Said gas stream comprising oxygen is produced in the following way. A gas stream comprising air of ambient temperature and pressure is fed to an air separation unit (ASU). The ASU is operated such that the following 2 streams leave the ASU: 1) a gas stream comprising nitrogen having a temperature of 40° C. and a pressure of 20 bar (which nitrogen can be subsequently stored); and 2) a gas stream comprising oxygen (purity of 99.5 mole %; 0.5 mole % of nitrogen) having a temperature of 40° C. and a pressure of 10 bar.

Said gas stream comprising methane and said gas stream comprising oxygen form a combined gas stream comprising methane and oxygen inside the OCM reactor, which combined gas stream comprises 80 mole % of methane and 20 mole % of oxygen (methane:oxygen molar ratio=4). The OCM reactor contains a methane oxidative coupling (OCM) catalyst and is operated under OCM conditions, including a temperature in the range of from 750 to 900° C. and a pressure of 10 bar. The conversion of methane is 35%, the selectivity to ethylene is 66.7% and the selectivity to ethane is 33.3%.

In Example A and in Comparative Example B, a product stream comprising 53.2 mole % of methane, 8.6 mole % of oxygen, 4.8 mole % of ethane, 9.6 mole % of ethylene and 23.9 mole % of water leaves the OCM reactor. Said product stream is cooled to a temperature of 40° C., thereby condensing out the water which is then separated. Any remaining water in said product stream is removed in a drying unit. After said water removal, said product stream is a gas stream comprising 69.8 mole % of methane, 11.3 mole % of oxygen, 6.3 mole % of ethane and 12.6 mole % of ethylene.

In Example A, the OCM reactor product stream from which water is removed is fed to a first sorption and desorption unit, which comprises a sorption agent which has an affinity for methane which is lower than that for ethane which in turn is lower than that for ethylene. All of the ethylene and 50% of the ethane are sorbed by the sorption agent (ethylene rejection=0%; ethane rejection=50%). A gas stream comprising oxygen, methane and ethane, and having a temperature of 40° C. and a pressure of 10 bar, leaves the first sorption and desorption unit, which oxygen, methane and ethane are not sorbed by the sorption agent in the first sorption and desorption unit.

In Example A, the gas stream comprising oxygen, methane and ethane which leaves the first sorption and desorption unit, is fed at the same pressure (10 bar) to a second sorption and desorption unit, which comprises a sorption agent which has an affinity for methane which is lower than that for ethane. All of the ethane is sorbed by the sorption agent (ethane rejection=0%; oxygen and methane rejection=100%). A gas stream comprising oxygen and methane, and having a temperature of 40° C. and a pressure of 10 bar, leaves the second sorption and desorption unit, which oxygen and methane are not sorbed by the sorption agent in the second sorption and desorption unit.

After some time, in Example A, the feed of the gas stream to the first sorption and desorption unit is stopped and the pressure in said unit is reduced from 10 bar to 1 bar, thereby inducing a first desorption step. The sorbed components (ethylene and ethane) subsequently become desorbed from the sorption agent and leave the first sorption and desorption unit at a temperature of 40° C. and a pressure of 1 bar. In Example A, the latter gas stream is advantageously enriched in ethylene as compared to the gas stream that is fed to the first sorption and desorption unit: the gas stream leaving the first sorption and desorption unit upon desorption comprises all of the ethylene and 50% of the ethane from the product stream.

After some time, in Example A, the feed of the gas stream to the second sorption and desorption unit is stopped and the pressure in said unit is reduced from 10 bar to 1 bar, thereby inducing a second desorption step. The sorbed components (ethane) subsequently become desorbed from the sorption agent and leave the second sorption and desorption unit at a temperature of 40° C. and a pressure of 1 bar. The gas stream leaving the second sorption and desorption unit upon desorption comprises 50% of the ethane from the product stream.

In Example A, the gas stream comprising ethylene and ethane which leaves the first sorption and desorption unit upon desorption is compressed to 16 bar by a compressor comprising 3 compression stages and then cooled to a temperature of −32° C. in two parallel heat exchangers utilizing the low temperature of the top and bottom streams coming from below-described distillation column A. Then said stream is fed to a distillation column having 99 theoretical stages, hereinafter referred to as distillation column A, and distilled, resulting in a top stream comprising ethylene and having a temperature of −38° C. and a pressure of 15 bar and in a bottom stream comprising ethane and having a temperature of −16° C. and a pressure of 16 bar. Said top and bottom streams are used to cool the feed streams in order to minimize condenser duty in distillation column A, which is provided by a propane refrigeration cycle

In Comparative Example B, the OCM reactor product stream from which water is removed is compressed to 31 bar by a compressor comprising 3 compression stages and then cooled to a temperature of −73° C. in three parallel heat exchangers utilizing the low temperature of the top stream coming from below-described distillation column B and the top and bottom streams coming from below-described distillation column C. Then said stream is fed to a distillation column having 22 theoretical stages, hereinafter referred to as distillation column B, and distilled, resulting in a top stream comprising oxygen and methane and having a temperature of −99° C. and a pressure of 31 bar and in a bottom stream comprising ethane and ethylene and having a temperature of −5° C. and a pressure of 31 bar. Said top stream is used to cool the feed streams in order to minimize condenser duty in distillation column B, which is provided by a cascaded ethylene-propane refrigeration cycle.

In Comparative Example B, the temperature and pressure of the bottom stream comprising ethane and ethylene coming from distillation column B are reduced to −30° C. and 16 bar by expansion, respectively. Then said stream is fed to a distillation column having 99 theoretical stages, hereinafter referred to as distillation column C, and distilled, resulting in a top stream comprising ethylene and having a temperature of −38° C. and a pressure of 15 bar and in a bottom stream comprising ethane and having a temperature of −16° C. and a pressure of 16 bar. Said top and bottom streams are used to cool the feed streams in order to minimize condenser duty in distillation column B, which is provided by a cascaded ethylene-propane refrigeration cycle.

Example C, also exemplifying the 1^{st} embodiment of the present invention (just like Example A), is performed in the same way as Example A, with the exception that a gas stream comprising air (that is to say, nitrogen and oxygen instead of oxygen alone), and having a temperature of 40° C. and a pressure of 10 bar, is fed to the OCM reactor. A product stream comprising 43.6 mole % of nitrogen, 30.0 mole % of methane, 4.8 mole % of oxygen, 2.7 mole % of ethane, 5.4 mole % of ethylene and 13.5 mole % of water leaves the OCM reactor. After the water removal, said product stream is a gas stream comprising 50.4 mole % of nitrogen, 34.7 mole % of methane, 5.5 mole % of oxygen, 3.1 mole % of ethane and 6.2 mole % of ethylene.

In Example C, the nitrogen rejection in the first and second sorption and desorption units is 100%. The gas stream comprising oxygen, nitrogen and methane which leaves the second sorption and desorption unit upon desorption is fed at the same pressure (10 bar) to a third sorption and desorption unit, which comprises a sorption agent which has an affinity for nitrogen which is lower than that for methane. All of the methane is sorbed by the sorption agent (methane rejection=0%; oxygen and nitrogen rejection=100%). A gas stream comprising oxygen and nitrogen, and having a temperature of 40° C. and a pressure of 10 bar, leaves the third sorption and desorption unit, which oxygen and nitrogen are not sorbed by the sorption agent in the third sorption and desorption unit.

After some time, in Example C, the feed of the gas stream to the third sorption and desorption unit is stopped and the pressure in said unit is reduced from 10 bar to 1 bar, thereby inducing a third desorption step. The sorbed components (methane) subsequently become desorbed from the sorption agent and leave the third sorption and desorption unit at a temperature of 40° C. and a pressure of 1 bar. The gas stream leaving the third sorption and desorption unit upon desorption comprises 100% of the methane from the product stream.

Comparative Example D is performed in the same way as Example C, with the exception that the OCM reactor product stream from which water is removed is compressed to 31 bar by a compressor comprising 3 compression stages and then cooled to a temperature of −111° C. in four parallel heat exchangers utilizing the low temperature of the top and bottom streams coming from below-described distillation column E and the top and bottom streams coming from distillation column C. Then said stream is fed to a distillation column having 22 theoretical stages, hereinafter referred to as distillation column D, and distilled, resulting in a top stream comprising nitrogen, oxygen and methane and having a temperature of −121° C. and a pressure of 31 bar and in a bottom stream comprising ethane and ethylene and having a temperature of −5° C. and a pressure of 31 bar. In Comparative Example D, the bottom stream comprising ethane and ethylene coming from distillation column D is separated in the same way using distillation column C as described above for Comparative Example B.

Further, in Comparative Example D, the top stream comprising nitrogen, oxygen and methane coming from distillation column D is expanded to a pressure of 17.5 bar and then cooled to a temperature of −137° C. in a heat exchanger utilizing the low temperature of the top stream coming from below-described distillation column E. Then said stream is fed to a distillation column having 22 theoretical stages, hereinafter referred to as distillation column E, and distilled, resulting in a top stream comprising nitrogen and oxygen and having a temperature of −159° C. and a pressure of 17 bar and in a bottom stream comprising methane and having a temperature of −111° C. and a pressure of 17 bar. Said top and bottom streams are used to cool the feed streams in order to minimize condenser duty in distillation columns D and E, which is provided by a cascaded methane-ethylene-propane refrigeration cycle.

In Table 1 below, the reflux ratios and the distillate-to-feed ratios needed to achieve the above separations in distillation columns A, B, C and D in Examples A and C and Comparative Examples B and D are mentioned. By said “reflux ratio”, reference is made to the molar ratio of the molar flow rate of the “reflux stream”, which is that part of the stream that leaves the condenser at the top of the distillation column which is sent back to that column, divided by the molar flow rate of the “distillate”, which is that part of the stream that leaves the condenser at the top of the distillation column which is not sent back to that column. By said “distillate-to-feed ratio”, reference is made to the molar ratio of the molar flow rate of said “distillate” divided by the molar flow rate of the feed stream that is fed to that column (the “feed”).

TABLE 1

| | Distillation | | Distillate-to- |
|---|---|---|---|
| Example(s) | column | Reflux ratio | feed ratio |
| A + C | A | 2.5 | 0.79 |
| B* | B | 3.0 | 0.65 |
| B* + D* | C | 1.0 | 0.73 |
| D* | D | 0.3 | 0.89 |
| D* | E | 2.3 | 0.74 |

*comparative

In all of the (Comparative) Examples, methane containing streams separated in the distillation columns may be recycled to the OCM reactor at 10 bar. The temperature reduction by reducing the pressure of such recycle methane containing streams to 10 bar, as well as the temperature reduction by reducing the pressure of nitrogen and oxygen containing top (vent) streams to atmospheric pressure, are utilized to cool the feed streams to the distillation columns and in this way the condenser duty provided by refrigeration is reduced.

In Table 2 below, the compression and refrigeration energy needed to convert methane into ethylene and to separately recover methane and ethylene from the product stream is included for all of Examples A and C and Comparative Examples B and D. Said energy is expressed as kilowatt hour (“kWh”; 1 kWh=3.6 megajoules) per kilogram (kg) of ethylene.

TABLE 2

| | | kWh/kg of |
|---|---|---|
| Ex. | Configuration | ethylene |
| A | O₂ [no N₂] | 2.21 |
| | 1^{st} PSA [100% methane + O₂ rejection + 50% | |
| | ethane rejection] | |
| | 1 distillation separating desorbed ethylene | |
| | and ethane | |
| | 2^{nd} PSA [100% methane + O₂ rejection] | |
| B* | O₂ [no N₂] + distillation only [no PSA] | 2.77 |
| C | air [O₂ + N₂] | 1.13 |
| | 1^{st} PSA [100% methane + O₂ + N₂ rejection + | |
| | 50% ethane rejection] | |
| | 1 distillation separating desorbed ethylene | |
| | and ethane | |
| | 2^{nd} PSA [100% methane + O₂ + N₂ rejection] | |
| | 3^{rd} PSA [100% N₂ rejection] | |
| D* | air [O₂ + N₂] + distillation only [no PSA] | 3.35 |

*comparative

From Table 2 above, it surprisingly appears that the energy needed to convert methane into ethylene (and optionally ethane, as in the 1^{st} embodiment of the present invention) and to separately recover methane and ethylene from the product stream is advantageously lowest in case the process of the present invention is carried out. That is, in all of Examples A and C, which exemplify the process of the present invention wherein in the OCM reaction step oxygen or air is used and in the subsequent product separation steps multiple sorption and desorption methods (in said Examples: PSA methods) are applied, said energy is advantageously lower than the energy needed to effect the same in those cases wherein sorption and desorption methods are not applied after the OCM reaction step, but only distillation steps are performed (as in Comparative Examples B and D), both when only oxygen (no nitrogen) is used in the OCM reaction step (Example A and Comparative Example B) and when air is used in the OCM reaction step (Example C and Comparative Example D). In the latter case (air), the advantageous different energy effect obtained with the process of the present invention is surprisingly largest.

Thus, surprisingly, this advantageous different energy effect obtained with the process of the present invention, as compared to the processes wherein only distillation steps are performed, is even obtained in cases where the first sorption and desorption steps are followed by 1 distillation step (Examples A and C) to recover the ethane and ethylene.

1. Process for the oxidative coupling of methane to one or more C2+ hydrocarbons, comprising
a reaction step which comprises subjecting a gas stream comprising methane and oxygen to methane oxidative coupling conditions resulting in a gas stream comprising methane, ethane and ethylene;
a first sorption step which comprises contacting the gas stream comprising methane, ethane and ethylene resulting from the reaction step with a sorption agent which has an affinity for methane which is lower than that for ethane which in turn is lower than that for ethylene, resulting in sorption of ethane and ethylene by the sorption agent and in a gas stream comprising methane and ethane;
a first desorption step which comprises desorbing ethane and ethylene sorbed in the first sorption step resulting in a gas stream comprising ethane and ethylene;
a second sorption step which comprises contacting the gas stream comprising methane and ethane resulting from the first sorption step with a sorption agent which has an affinity for methane which is lower than that for ethane, resulting in sorption of ethane by the sorption agent and in a gas stream comprising methane;
a second desorption step which comprises desorbing ethane sorbed in the second sorption step resulting in a gas stream comprising ethane; and
optionally a recycle step which comprises recycling the gas stream comprising methane resulting from the second sorption step to the reaction step.

2. Process according to claim 1, comprising
a reaction step which comprises subjecting a gas stream comprising methane and air to methane oxidative coupling conditions resulting in a gas stream comprising nitrogen, methane, ethane and ethylene;
a first sorption step which comprises contacting the gas stream comprising nitrogen, methane, ethane and ethylene resulting from the reaction step with a sorption agent which has an affinity for nitrogen which is lower than that for methane which in turn is lower than that for ethane which in turn is lower than that for ethylene, resulting in sorption of ethane and ethylene by the sorption agent and in a gas stream comprising nitrogen, methane and ethane;
a first desorption step which comprises desorbing ethane and ethylene sorbed in the first sorption step resulting in a gas stream comprising ethane and ethylene;
a second sorption step which comprises contacting the gas stream comprising nitrogen, methane and ethane resulting from the first sorption step with a sorption agent which has an affinity for nitrogen which is lower than that for methane which in turn is lower than that for ethane, resulting in sorption of ethane by the sorption agent and in a gas stream comprising nitrogen and methane;
a second desorption step which comprises desorbing ethane sorbed in the second sorption step resulting in a gas stream comprising ethane.

3. Process according to claim 2, additionally comprising
a third sorption step which comprises contacting the gas stream comprising nitrogen and methane resulting from the second sorption step with a sorption agent which has an affinity for nitrogen which is lower than that for methane, resulting in sorption of methane by the sorption agent and in a gas stream comprising nitrogen;
a third desorption step which comprises desorbing methane sorbed in the second sorption step resulting in a gas stream comprising methane; and
optionally a recycle step which comprises recycling the gas stream comprising methane resulting from the third desorption step to the reaction step.

4. Process according to claim 1, additionally comprising
a distillation step which comprises distilling the gas stream comprising ethane and ethylene resulting from the first desorption step, resulting in a top stream comprising ethylene and a bottom stream comprising ethane.

5. Process for the oxidative coupling of methane to one or more C2+ hydrocarbons, comprising
a reaction step which comprises subjecting a gas stream comprising methane and air to methane oxidative coupling conditions resulting in a gas stream comprising nitrogen, methane and ethylene;
a first sorption step which comprises contacting the gas stream comprising nitrogen, methane and ethylene resulting from the reaction step with a sorption agent which has an affinity for nitrogen and methane which is lower than that for ethylene, resulting in sorption of ethylene by the sorption agent and in a gas stream comprising nitrogen and methane;
a first desorption step which comprises desorbing ethylene sorbed in the first sorption step resulting in a gas stream comprising ethylene;
a second sorption step which comprises contacting the gas stream comprising nitrogen and methane resulting from the first desorption step with a sorption agent which has an affinity for nitrogen which is lower than that for methane, resulting in sorption of methane by the sorption agent and in a gas stream comprising nitrogen;
a second desorption step which comprises desorbing methane sorbed in the second sorption step resulting in a gas stream comprising methane; and
optionally a recycle step which comprises recycling the gas stream comprising methane resulting from the second desorption step to the reaction step.

6. Process according to claim 1, wherein desorption in the first, second and optional third desorption steps is effected by reducing the pressure.

7. Process according to claim 6, wherein the pressure in the first, second and optional third sorption step is in the range of from 5 to 30 bar, preferably 5 to 15 bar, more preferably 7 to 13 bar, and the pressure in the first, second and optional third desorption step is in the range of from 0.1 to 3 bar, preferably 0.5 to 2 bar.

8. Process according to claim 7, wherein in the reaction step oxygen or air is fed at a pressure in the range of from 5 to 15 bar, preferably 7 to 13 bar.
